(19) United States
(12) Patent Application Publication
(10) Pub. No.: US 2024/0157629 A1
(43) Pub. Date: May 16, 2024

(54) 3D BIOPRINTER WITH CONTINUOUS WORKFLOW CAPABILITY

(71) Applicants: Hector Martinez, Gothenburg (SE); Patrick Thayer, Hudson, MA (US); Erik Gatenholm, Gothenburg (SE); Holger Eickhoff, Syke (DE); Cellink Bioprinting AB, Gothenburg (SE)

(72) Inventors: Hector Martinez, Gothenburg (SE); Patrick Thayer, 50 Walnut Street, MA (US); Erik Gatenholm, Gothenburg (SE); Holger Eickhoff, Syke (DE)

(21) Appl. No.: 18/550,279

(22) PCT Filed: Mar. 14, 2022
(86) PCT No.: PCT/US22/20148

§ 371 Date: Sep. 12, 2023

Related U.S. Application Data
(60) Provisional application No. 63/160,159, filed on Mar. 12, 2021.

(51) Int. Cl. B29C 64/106; B29C 64/182; B29C 64/209; B33Y 30/00; B33Y 40/20

(52) U.S. Cl. CPC B29C 64/106; B29C 64/182; B29C 64/209; B33Y 30/00; B33Y 40/20

(57) ABSTRACT

Bioprinters use biological inks (i.e. bioinks) to produce biomaterials such as cell cultures (including 3D cell cultures), living tissues, and organs. Bioprinters as described by the present invention include those capable of performing high-throughput industrial scale assays, applications, and development while maintaining precision.

### CROSS-REFERENCE TO RELATED APPLICATIONS

This application relies on the disclosure of and claims priority to and the benefit of the filing date of U.S. Provisional Application No. 63/160,159, filed Mar. 12, 2021, which is hereby incorporated by reference herein in its entirety.

### BACKGROUND OF THE INVENTION

### Field of the Invention

The present invention relates to the field of bioprinters, specifically those capable of performing high-throughput industrial scale assays, applications, and development while maintaining precision. Bioprinters use biological inks (i.e. bioinks) to produce biomaterials such as cell cultures (including 3D cell cultures), living tissues, and organs.

### SUMMARY OF THE INVENTION

Embodiments of the present invention provide systems and methods related to bioprinters and bioprinting workflows. Exemplary aspects of embodiments of the invention are described in more detail below; however, these examples are merely examples and variations of such are also within the scope of the invention.

Aspects of embodiments of the invention include Aspect 1, which is a bioprinter comprising: one or more or multiple printheads moveable/positionable in x-, y- and/or z-directions, which printheads are capable of depositing one or more materials; one or more or multiple cartridges/containers operably communicable with one or more of the printheads for containing one or more of the materials (such as one or more of cells, spheroids, and/or bioinks (such as one or more media, including growth media, growth factors, buffers, crosslinking agents/buffers, cell buffers etc.); optionally, one or more or multiple bulk liquid dispensers; one or more dispensing system for dispensing one or more of the materials from one or more of the printheads, optionally with a dispensing resolution down to nanoliters or picoliters; one or more printbed moveable/positionable in x-, y-, and/or z-directions; optionally, one or more temperature control systems for controlling temperature of one or more of the printheads (e.g., in the range of from −2° C. up to 350° C.) and/or one or more of the printbeds (e.g., in the range of −20° C. up to 120° C.); optionally, one or more robotic arm for plate/lid positioning (such as adding, removing, exchanging and/or positioning lids and/or well plates and/or slides) or any substrate on which material is bioprinted, such as slides) and optionally wherein the plate/lid positioning is capable of a positioning resolution of down to 1 μm; one or more control system for controlling movement/position and/or operation of one or more of the printheads, printbeds, dispensing system, and/or robotic arm; optionally, one or more control system for performing/controlling crosslinking, washing, add/or adding media to the constructs, or for adding one or more cells or spheroids to the media; wherein one or more of the control systems are configured to provide for an automated workflow, such as without human intervention, for printing into 25 or more well plates, such as any number of plates, automatically and/or consecutively; optionally, a hermitically sealed or sealable housing (such as with humidity, temperature, atmospheric control and/or dew point control).

Aspect 2 is a bioprinter comprising: one or more or multiple printheads moveable/positionable in x-, y- and/or z-directions, which printheads are capable of depositing one or more materials; one or more or multiple cartridges/containers operably communicable with one or more of the printheads; one or more dispensing system for dispensing one or more of the materials from one or more of the printheads with a dispensing resolution down to nanoliters or picoliters; a hermitically sealed or sealable housing; one or more printbed moveable/positionable in x-, y-, and/or z-directions; one or more temperature control systems for controlling temperature of one or more of the printheads and/or one or more of the printbeds; one or more robotic arm for plate/lid positioning with a resolution of down to 11 μm; one or more control system for controlling movement/position and/or operation of one or more of the printheads, printbeds, dispensing system, and/or robotic arm; one or more control system for performing/controlling crosslinking, washing, add/or adding media to the constructs, or for adding one or more cells or spheroids to the media; and wherein one or more of the control systems are configured to provide for an automated workflow for printing into 25 or more well plates, such as any number of plates, automatically and/or consecutively.

Aspect 3 is the bioprinter of Aspect 1 or 2, wherein the automated workflow is provided by a set of computer-executable instructions capable of performing the workflow.

Aspect 4 is a method comprising: preparing one or more bioprinting construct through the deposition of one or more biomaterial or bioink in the same workflow in combination with other deposition modalities or workflow steps; wherein the same workflow refers to a set of computer-executable instructions for performing the dispensing and deposition without human intervention, such as continuously and automatically; wherein the droplet dispensing comprises dispensing of pico- or nano-scale sized droplets; and/or wherein in the same workflow large volumes can be dispensed such as and including large volumes relating to washing buffer, cell media, or crosslinking solution.

Aspect 5 is the method of Aspect 4, comprising the combination of bioprinting and construct or scaffold specific targeted dosing.

Aspect 6 is the method of Aspect 4 or 5, comprising printing/depositing/dispensing bioink and then printing, depositing, and/or positioning one or more cell or spheroid within the printed bioink.

Aspect 7 is the method of any of Aspect 4-6, wherein the bioink is printed separately from the cell(s) or spheroid(s).

Aspect 8 is the method of any of Aspects 4-7, wherein the cell(s) and/or spheroid(s) are printed or deposited with a degree of precision and/or patterning with a resolution of down to 1 μm.

Aspect 9 is the method of any of Aspects 4-8, comprising printing, depositing, and/or positioning one or more cell or spheroid then printing/depositing/dispensing one or more bioink, drug, and/or growth factor.

Aspect 10 is the method of any of Aspects 4-9, wherein the printing, depositing, and/or dispensing is performed such that a resolution in the picoliter range is achieved.

Aspect 11 is the method of any of Aspects 4-10, wherein the printing, depositing, and/or dispensing results in drop/spot sizes of about 1 micron to about 500 microns.

Aspect 12 is the method of any of Aspects 4-11, wherein the printing, depositing, and/or dispensing is performed with a precision in the micrometer range.

Aspect 13 is a bioprinter configured to perform the method of any of Aspects 4-12.

Aspect 14 is a bioprinter comprising: one or more or multiple print heads; one or more printbed; one or more liquid dispenser.

Aspect 15 is the bioprinter of Aspect 14, wherein the one or more or multiple printheads and/or the one or more printbed is moveable/positionable in x-, y-, and/or z-directions with a precision in the micrometer range.

Aspect 16 is the bioprinter of aspect 14 or 15, wherein the precision is as low as 1 micrometer.

Aspect 17 is the bioprinter of any of Aspects 14-16, wherein the one or more printhead is capable of depositing bioink and/or spheroids.

Aspect 18 is the bioprinter of any of Aspects 14-17, further comprising a positioning arm.

Aspect 19 is the bioprinter of any of Aspects 14-18, wherein the positioning arm is capable of performing one or more function to facilitate bioprinting, crosslinking, washing, and/or dispensing of one or more solid and/or liquid.

Aspect 20 is the bioprinter of any of Aspects 14-19, wherein the positioning arm is capable of performing one or more function to facilitate bioprinting, crosslinking, washing, and/or dispensing of one or more solid and/or liquid without user intervention.

Aspect 21 is the bioprinter of any of Aspects 14-20, wherein the one or more function is placement of a printing surface onto the printbed, or removal or placement of a lid from/on a printing surface.

Aspect 22 is the bioprinter of any of Aspects 14-21, wherein the liquid dispenser is capable of dispensing one or more liquid and/or bioink with a resolution of at least 1 picoliter.

Aspect 23 is the bioprinter of any of Aspects 14-22, wherein the bioprinter is capable of printing a construct using spheroids and/or single cells.

Aspect 24 is the bioprinter of any of Aspects 14-23, wherein the bioprinter is capable of printing spheroids and/or cells separately from one another and/or separately from bioinks.

Aspect 25 is the bioprinter of any of Aspects 14-24, wherein the bioprinter is capable of dispensing growth factors, drugs, and/or therapies.

Aspect 26 is the bioprinter of any of Aspects 14-25, wherein the one or more liquid dispenser is capable of dispensing drop(s)/spot(s) ranging from 1 micron to 500 micron or more (or any range in between), and/or with an accuracy (e.g., absolute position) in the micrometer range.

Aspect 27 is the bioprinter of any of Aspects 14-26, wherein the dispensing can be performed with precision in the micrometer range, such as a dispensing precision or repeatability in the range of less than 3 micrometers, for example, less than 2 micrometers, or less than 1 micrometer.

Aspect 28 is the bioprinter of any of Aspects 14-27, wherein the one or more print head is/are capable of depositing one or more bioink and one or more spheroid.

Aspect 29 is the bioprinter of any of Aspects 14-28, wherein the one or more print head, one or more liquid dispenser, and/or one or more printbed are positionable with up to 1 μm precision.

Aspect 30 is the bioprinter of any of Aspects 14-29, further comprising one or more camera(s).

Aspect 31 is the bioprinter of any of Aspects 14-30, wherein the one or more camera(s) is capable of providing high definition video and/or images.

Aspect 32 is the bioprinter of any of Aspects 14-31, wherein the bioprinter is capable of providing camera quality control and/or quality assurance.

Aspect 33 is the bioprinter of any of Aspects 14-32, further comprising one or more onboard display or user interface.

Aspect 34 is the bioprinter of any of Aspects 14-33, wherein the one or more onboard display or user interface is capable of displaying images and/or video from the one or more camera(s).

### BRIEF DESCRIPTION OF THE DRAWINGS

The accompanying drawings illustrate certain aspects of implementations of the present disclosure, and should not be construed as limiting. Together with the written description the drawings serve to explain certain principles of the disclosure.

FIG. 1 is an illustration of a bioprinter according to an embodiment of the invention.

FIG. 2 is an illustration of various bioprinter print heads and a printbed according to embodiments of the invention.

FIG. 3 is a flow chart of a method of bioprinting according to an embodiment of the invention.

FIG. 4 is a flow chart of a method of bioprinting and crosslinking according to an embodiment of the invention.

FIG. 5 is a flow chart of a method of bioprinting and dispensing cell media according to an embodiment of the invention.

FIG. 6 is a flow chart of a method of bioprinting, crosslinking, and washing according to an embodiment of the invention.

### DETAILED DESCRIPTION OF VARIOUS EMBODIMENTS OF THE INVENTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention. Any one or more methods in whole or part can be incorporated in any one or more of the systems to provide a desired workflow function, and any one or more of the systems can be used to perform any one or more of the methods in whole or in part.

In embodiments of the present invention, the inventive 3D bioprinters provide one or more of the following functionalities/capabilities:

1) A combination of single cell/spheroid printing with biomaterial deposition. This novel approach enables new workflows to study cell-material interactions and build constructs with higher cell precision and patterning than other approaches.

2) Fully automated workflows. Typically, a user has to manually crosslink, wash, add media, etc. to the constructs post printing. With the inventive 3D bioprinter, one or more or all of these previous steps can be automated, which streamlines workflows and minimizes the chance of user error.

3) Ability to combine nanoscale droplet dispensing with biomaterial deposition in the same workflow. This enables printing in combination with targeted dosing.

The inventive bioprinters can be used for developing/fabricating 3D cell culture models, for high throughput testing using such models, and to provide improvements in the field of 3D bioprinting, which field includes such technologies as described in, for example, WO2020/165322, WO2019/109127, WO2019/246623, WO2017/109394, WO2017/040675, WO2015/148646, U.S. Pat. Nos. 9,315,043, 8,931,880, US2020/0139623, US2020/0070421, US2019/0344500, US2019/0016052, US2018/0326665, US2018/0281280, US2016/0344500, US2016/0288414, US2016/0243618, US2015/0375453, US2015/0105891, and US2011/024699, which references are each incorporated by reference herein in their entireties.

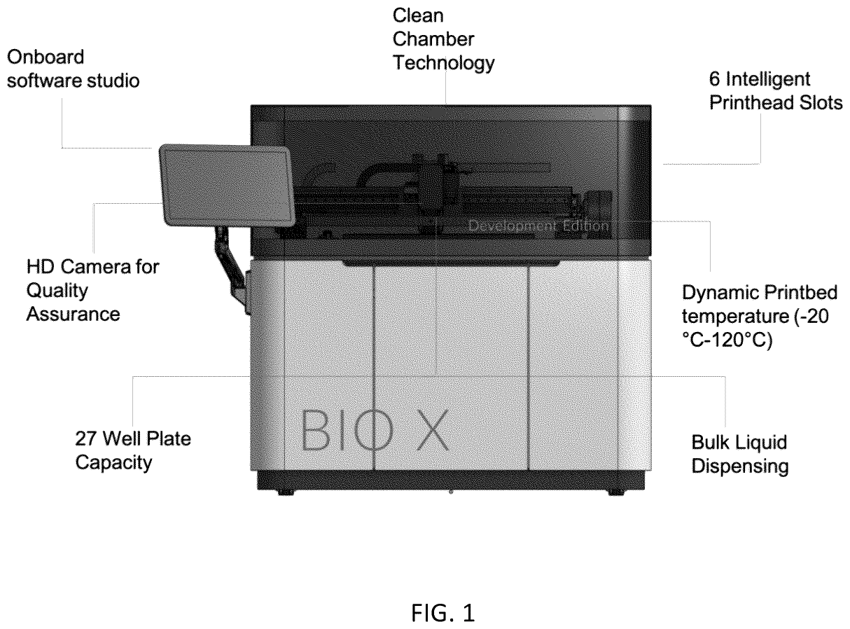

FIG. 1 shows an example bioprinter according to an embodiment of the present invention. The bioprinter comprises one or more of: a clean chamber, up to 6 intelligent printhead slots, a temperature controlled printbed, one or more dispensers for liquid dispensing, a capacity to hold up to 27 well plates, an HD camera for quality assurance, an onboard software studio, and a user interface.

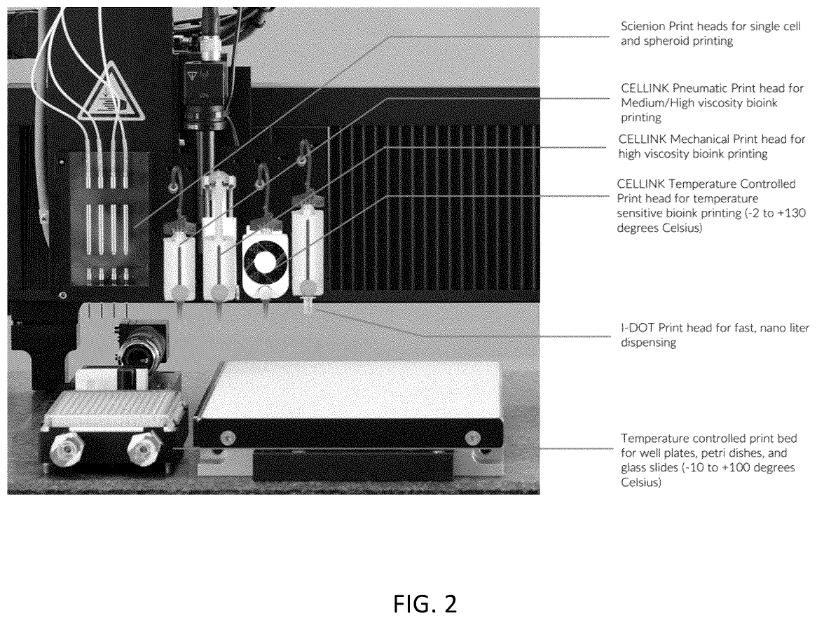

FIG. 2 shows an example of a bioprinter comprising multiple printheads according to an embodiment of the invention. The bioprinters for example can comprise one or more of the following features/functionalities, including clean chamber technology, semi-automation, automation, PDCs (piezo dispense capillaries or pico dispense capillaries), NDCs (nano dispense capillaries), i-DOT source wells, spheroid printheads, BIO X iPH technologies, i-DOT printheads, sciDROP PICO printheads, computer vision, imaging modules, cooling solutions, sciDROP NANO printheads, and/or cell dispensing and dosing in a single unit.

Embodiments can comprise any combination of such features. For example, embodiments can comprise one or more of the following: one or more printheads for single cell and/or spheroid printing, one or more pneumatic printheads for medium/high viscosity bioink printing, one or more mechanical printheads for high viscosity bioink printing, one or more temperature controlled printheads (for example, −2° C. to 350° C.) for temperature sensitive bioink printing, one or more i-DOT printheads for fast, nanoliter dispensing, and/or a temperature controlled printbed (for example, −20° C. to 120° C.) for supporting well plates, petri dishes, and/or glass slides.

In embodiments, the inventive 3D bioprinters can comprise up to 12 or more bioprinting toolheads, such as from 1, 2, 3, 4, 5, 6, 7, 8, 9, 10, 11, or 12 printing toolheads, or more. One or more printheads can provide any one or more of the following features/functionalities, including pneumatic operation, temperature control of the printhead or deposition material, thermoplastic materials, EMD (electro-magnetic droplet) capabilities, FDM (fused deposition modeling) capabilities, standard syringes, cooled syringe printheads, bulk droplet dispensers (for example, 1 μL to mL range), single-cell printheads, spheroid printheads, large volume dispensers (such as up to 50 mL or more), or can comprise screw drive capabilities.

The toolheads/printheads can be modular (i.e. interchangeable), such that one or more toolheads can be removed, added, replaced, or exchanged as desired to perform a particular assay and/or a particular build.

Exemplary 3D bioprinters can include any one or more of the following features:

The plate or slide capacity is for example a 27 plate capacity or 140 slide capacity, such as accommodating from 1-27 well plates (which may be referred to as microplates) or from 1-140 slides, or more, or in embodiments any number of plates or slides. Embodiments comprise plate and/or slide manipulation capacity, where for example well plates and/or slides can be automatically or manually removed, added, replaced, positioned, uncapped, and/or recapped to accommodate high-throughput assays and/or to provide for continuous operation of the 3D bioprinter. Printing can be performed on any surface, including on slides, well plates, microplates or custom surfaces. In embodiments, the printing surface can be automatically or manually moved, covered (e.g. with a lid), and/or placed in a location where it can be removed or transferred to another piece of equipment.

A typical maximum build dimension capable of being provided can be up to 82.5 cm in length, up to 36 cm in width, and up to 15 cm in height. Depending on the particular application and/or build and/or type of construct being prepared, other dimensions are also possible, including larger dimensions, only confined by the size of the overall housing of the 3D printer which can be configured/sized accordingly.

Bulk liquid dispensing capabilities are also provided and can be provided by 1, 2, or 3 separate liquid sources. In other embodiments, the system may comprise up to 10 separate liquid sources. Bulk liquid dispensing sources can hold liters of liquid, such as up to 1 L, up to 2 L, up to 5 L, or up to 10 L.

In embodiments, the bioprinter comprises one or more large volume dispenser(s) capable of dispensing volumes of up to about 500 mL, such as up to about 1 mL, 5 mL, 10 mL, 25 mL, 50 mL, 100 mL, or 250 mL.

The bioprinters have photocuring capabilities, which can be provided by one or more UV or visible light source, such as light source(s) providing UV or visible light. In embodiments, one or more wavelengths, such as 1, 2, 3, or 4 wavelengths, which can be the same or different wavelengths and/or can be provided by the same or different light sources, are customizable and chosen from a range of wavelengths in the range of from about 300 to 700 nm, such as 365 nm, 405 nm, 425 nm, or 525 nm. Crosslinking can be performed in any number of ways, including by one or more of ionic crosslinking, stereocomplex crosslinking, thermal crosslinking, photocrosslinking, enzymatic gelation, and/or Click chemistry.

The 3D bioprinters can be equipped with platform/printbed temperature control, which can provide platform/printbed temperatures ranging from −20° C. to 120° C. and/or the 3D bioprinters can be equipped with printhead temperature control to provide one or more printheads at a temperature of up to 250° C. Such temperature capabilities can be provided in a manner such that the temperature of the environment(s) within which the printbed and/or printhead(s) is controlled to be in the range of from −2° C. to 350° C., such as from 18° C. to 28° C.

In embodiments, one or more component(s) of the bioprinter is temperature controlled and is capable of being maintained at a temperature of about −20° C. to about 350° C., such as about −10° C., −5° C., −2° C., 0° C., 4° C., 10° C., 15° C., 20° C., 25° C., 28° C., 30° C., 35° C., 37° C., 40° C., 45° C., 50° C., 60° C., 70° C., 80° C., 90° C., 110° C., 130° C., 150° C., 175° C., 200° C., 225° C., 250° C., 275° C., 300° C., or 325° C. In embodiments, the one or more temperature-controlled components include the printhead (or bioprinting toolhead), dispenser(s), liquid reservoirs, and/or printbed. In embodiments, temperature is capable of being maintained at the desired temperature up to a range within ±1° C., whether the temperature being controlled is one or more of the printbed, the bioink, the printing platform or substrate, the well plate, the slides, and/or the environment in which the components of the 3D bioprinter and/or the construct being built and/or the assay being performed are exposed/subjected to.

Environmental controls can also include one or more of particle filters, sterile filters, humidity control, temperature control, and/or dew point control. In some embodiments, the 3D bioprinter comprises software to report summaries and graphs showing environmental parameters over a selected time period.

Spheroids or cells can be printed separately from one another and/or separately from bioinks. This enables the depositing of bioink and subsequently positioning of cells and/or spheroids at any position relative to the bioink, such as within, on and/or near the bioink. Similarly, a construct can be built using spheroids and/or single cells and subsequently growth factors and/or other bioinks can be applied, which enables one or more, which can be the same or different, concentrations of drugs, therapies, and/or growth factors to be used. In embodiments, multiple channels can be used to deliver spheroids and/or cells, such as two or three different channels for spheroids and/or two or three different channels for cells. The spheroids in the multiple channels, and/or the cells in the multiple channels, can be the same or different relative to the other channels. It is feasible that any number of channels could be employed depending on the needs of a particular application and/or assay, such as 1, 2, 3, 4, 5, or more channels.

Bioinks capable of being printed using embodiments of the inventive 3D bioprinter can include any one or more of hydrogel-based bioinks, polysaccharides, protein-based bioinks, dECM-based bioinks (e.g., decellularized bioinks), and/or synthetic polymer-based bioinks, including bioinks comprising one or more of alginate, gelatin, collagen, fibrin/fibrinogen, gellan gum, hyaluronic acid (HA), agarose, chitosan, silk, silk fibroin, decellularized extracellular matrix (dECM), poly(ethylene glycol) (PEG), PEG diacrylate (PEGDA), and Pluronics, gelatin-alginate composites, functionalized gelatin (GelMA), fibrinogen, fibrin and alginate, alginate and fibroblasts, cell aggregate based bioinks, and/or pellet-based bioinks. Examples of such bioinks are double network bioinks, biogum and botanical gum hydrogel bioinks, RGD conjugated polysaccharide bioinks with or without fibrin, and bioinks comprising cellulose nanofibrils with extracellular matrix components, such as any bioink described in WO2020/077118, US2021/0001009, US2019/0160203, and/or US2019/0209738, which are each incorporated by reference herein in their entireties.

Bioinks and/or media can include samples comprising aqueous solutions (e.g. oligonucleotides) and/or organic solvents, samples containing organic solvents like DMSO, DMF etc. and protein mixtures (e.g. lysates, allergens etc.), samples containing protein solutions and organic solvents like methanol, isopropanol, acetonitrile etc., and/or protein solutions and solgel samples.

Dispensing is capable of being performed down to the picoliter and/or nanoliter range, such as with a resolution of down to 1 pL, or down to 10 pL, such as with a dispensing resolution in the range of from about 1-10 pL, or from 10 pL to 1 nL, or from 1 nL to 10 nL, or below 10 nL, or from 10 nL to 1,000 nL, or any range in between. Embodiments can comprise dispensing/printing with piezo dispense capillary capability (or otherwise referred to as pico- or nano-dispense capillary) with a fixed drop volume for example ranging from 50-800 pL drops, such as from 100-150 pL, or from 150-220 pL, or from 220-300 pL, or form 300-360 pL, or from 360-440 pL, or from 440-520 pL, or from 520-600 pL, or from 600-800 pL, or from 100 pL to 1.0 mL, or from 1-100 nL drops, such as from 1-10 nL drops, or from 5-50 nL drops, etc.

Dispensing can be performed with drop/spot sizes ranging from 1 micron to 500 micron or more (or any range in between), and/or with an accuracy (e.g., absolute position) in the micrometer range, such as less than 10 micrometers, or less than 5 micrometers, or less than 1 micrometer, with resolutions of 1 micrometer or less, such as 0.5 micrometers, or 0.1 micrometers. In embodiments, the drop size achieved can be from about 1 micron to about 1,000 microns, such as about 5 microns, 10 microns, 20 microns, 50 microns, 100 microns, 125 microns, 175 microns, 200 microns, 250 microns, 325 microns, 375 microns, 450 microns, 550 microns, 600 microns, 750 microns, 800 microns, or 900 microns. Dispensing can be performed with precision in the micrometer range, such as a dispensing precision or repeatability in the range of less than 3 micrometers, for example, less than 2 micrometers, or less than 1 micrometer. For example, extrusion filaments can be deposited onto any substrate in the range of down to 1 micron apart.

Bioprinting can be performed at a rate of up to 1500 mm/s, such as from 1 mm/s to 1500 mm/s, or from 100 mm/s to 750 mm/s, or from 250 mm/s to 1250 mm/s, or from 500 mm/s to 1000 mm/s, and/or with a flow rate of up to 20 mL/min., such as from above 0 mL/min. to 10 mL/min., or from about 1 mL/min. to about 5 mL/min., or from about 2 mL/min. to about 8 mL/min., or any range in between.

The viscosity of the materials being printed can be in the range of up to 400 cP, or anywhere in the range of from 0.1-10,000 Pa·s, such as from 500-2,000 Pa·s, or from 0.2-10 Pa·s, or from 75-2,500 Pa·s, or from 5-100 Pa·s, or from 1.5-2 Pa·s. The dispensing/printing can be used to print dots, lines, bars, coatings, etc.

It is noted in particular that where a range of values is provided in this specification, each value between the upper and lower limits of that range is also specifically disclosed. The upper and lower limits of these smaller ranges may independently be included or excluded in the range as well. Where temperatures are given in this specification, the values are understood to be the indicated temperature ±1° C. For example, where a temperature of 4° C. is given, it is understood to mean any temperature between 3° C. and 5° C. inclusive. The term “about” is defined in this specification in the context of quantitative measurements to mean the indicated amount ±10%. For example, with a ±10% range, “about 1 mL” can mean 0.9-1.1 mL.

In embodiments, the 3D bioprinters are capable of performing automated quality control and/or quality assurance. In embodiments, camera quality control and/or quality assurance is provided. In some embodiments, one or more of the cameras is capable of providing high definition video and/or images. In some embodiments, an onboard display is capable of providing a software studio showing images and/or video from the camera(s).

The inventive bioprinters are configured to automate complete bioprinting workflows. Integrated photocuring toolheads, bulk liquid dispensers, and a positioning arm allows the user to bioprint constructs, crosslink, wash, and add media without user intervention. Nanoliter and picoliter dispensing capabilities allow the fabrication of constructs followed by dosing with growth factors, small molecules, and other therapies in preparation for characterization.

In embodiments, the bioprinters comprise one or more transfer unit (such as a robotic arm) to facilitate the movement and/or positioning of the bioprinted construct and/or printing surface. In embodiments, a robotic positioning arm is included for plate/lid positioning and to provide the function of adding, removing, or exchanging one or more objects, such as lids and/or well plates (or any substrate on which material is bioprinted, such as slides) and for positioning them within the workflows. The robotic positioning arm is capable of gripping, releasing, pushing, pulling, lifting, positioning and/or placing one or more of the same or different of such objects.

In embodiments, the printbed is stationary. In other embodiments the printbed is positionable in the x-, y-, and/or z-direction(s). In embodiments, the printbed comprises one or more fitting to secure the printing surface to prevent movement.

In embodiments, a cell mixer, such as a continuous or semi-continuous cell mixer, can be included to enable a continual bioprinting process. A cartridge loader/unloader can be included, which provides for automatic or semi-automatic loading/unloading of cartridges, such as when an empty cartridge of cells/spheroids and/or bioinks needs replaced.

In embodiments, the inventive bioprinters can precisely print any structure or material, such as lattices, filaments, droplets, spheroids, and/or single cells. To the best of the inventors' knowledge, this is the only biofabrication platform to incorporate both bioink and spheroid depositing capabilities, enabling novel workflows and flexibility in as say development. With up to 1 μm precision in positioning and fully automated bioink parameter characterization, repeatability is assured.

### EXAMPLES

The following examples are intended to be illustrative only. One or more method steps in the following examples may be added to, omitted from, or combined with method steps of a separate example, likewise any one or more components of a system can be combined. In embodiments, to obtain a system for a desired workflow, the components or functionalities can be linked together via a transfer unit or in operable communication with a transfer unit.

Example workflows the bioprinters are capable of performing include but are not limited to:
Workflow 1: print
Workflow 2: print and crosslink
Workflow 3: print and dispense media
Workflow 4: print, crosslink, and wash

### Example 1. Print

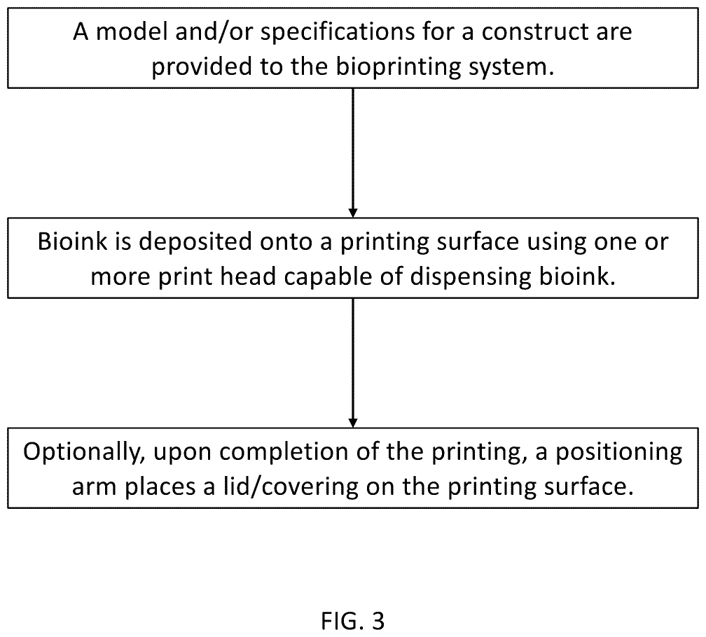

An example bioprinting workflow includes bioprinting a construct (FIG. 3). A model is uploaded to the bioprinter for printing and/or specifications for a construct are provided to the bioprinting system. Using one or more print head capable of dispensing bioink, bioink is deposited onto a printing surface, such as a 24 well plate, according to the model and/or specifications. The well plate optionally rests on a temperature-controlled printbed during the bioprinting process. Upon completion of the printing, the positioning arm places a well plate lid on the printing surface.

### Example 2. Print and Crosslink

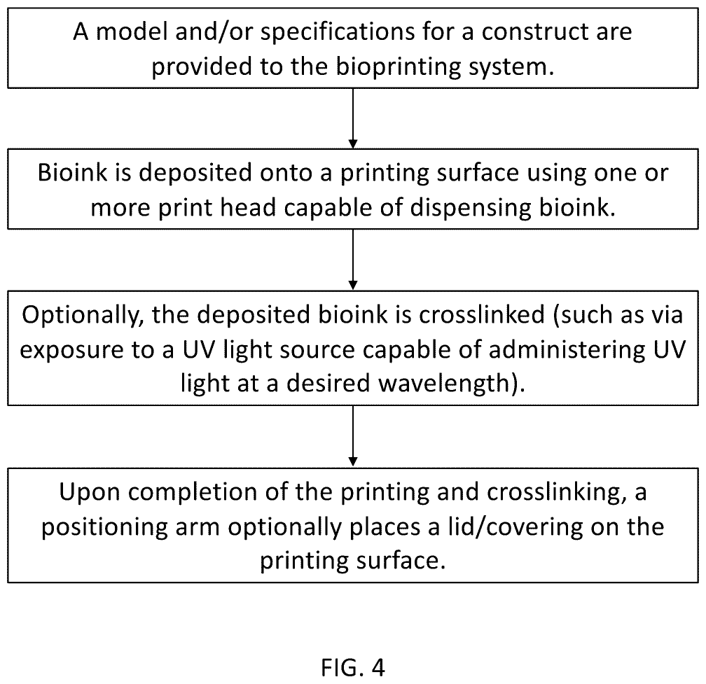

An example bioprinting workflow includes bioprinting a construct and crosslinking the construct (FIG. 4). Using one or more print head, bioink is deposited onto the printing surface, such as a glass slide. The glass slide rests on and/or is secured to the printbed during the bioprinting process. Using one or more print head, the bioink is deposited on the glass slide. After depositing the bioink, the construct is crosslinked via exposure to a UV light source capable of administering UV light at a desired wavelength, such as at a wavelength in the range of 300-700 nm. In embodiments, the crosslinking and/or curing can be performed as the bioink is being extruded from the printhead(s).

### Example 3. Print and Dispense Media

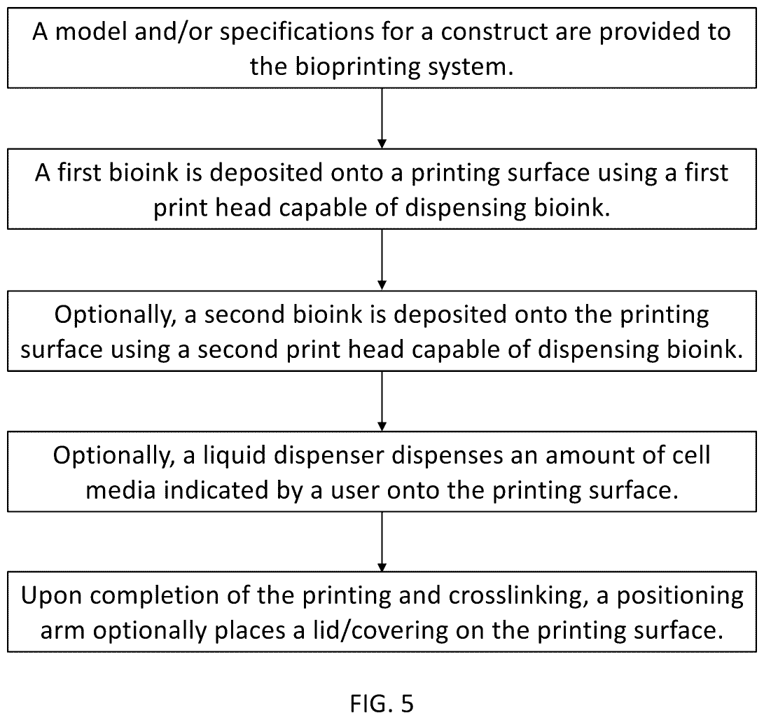

An example bioprinting workflow includes bioprinting a construct and dispensing media (FIG. 5). Using a first print head, a first bioink comprising a first cell type is deposited onto the printing surface, such as a petri dish. Next, using a second print head, a second bioink comprising a second cell type is deposited onto the printing surface. The print heads continue the depositing of the bioinks until printing of the desired construct is complete. In embodiments, the printing can alternate between printing one or more cell types and/or the printing can be performed to deposit one cell type and then another cell type. A liquid dispenser dispenses an amount of cell media indicated by a user onto the printing surface. In embodiments, the cells can be printed onto cell media and/or the cell media can be printed onto one or more cell types.

### Example 4. Print, Crosslink, and Wash

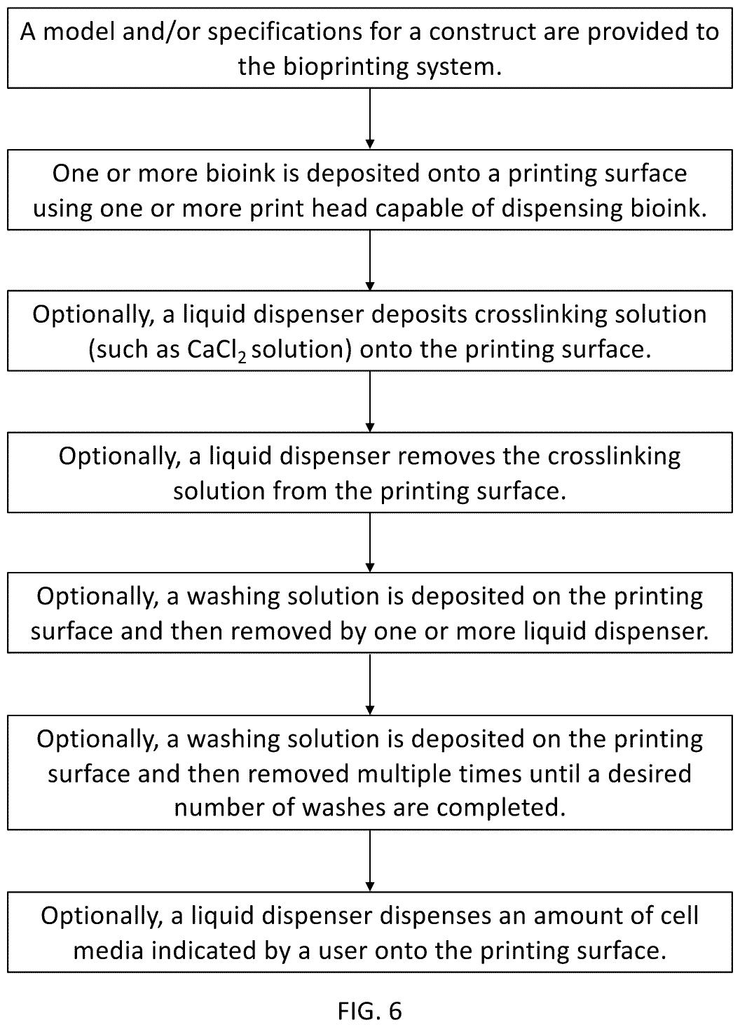

An example bioprinting workflow includes bioprinting a construct, including crosslinking and washing steps (FIG. 6). Using a first print head, a first bioink comprising a first cell type is deposited onto the printing surface, such as a petri dish. Next, using a liquid dispenser, a calcium chloride-containing crosslinking solution is deposited onto the printing surface, or the calcium chloride-containing or other crosslinking solution can be deposited before the cells. Crosslinking is allowed to occur. After crosslinking, one or more liquid dispenser removes the crosslinking solution and deposits a washing solution, such as cell media, onto the printing surface. The washing solution is removed and more washing solution is optionally deposited until a number of desired washing steps have been completed.

According to embodiments, any one or more of the printing, dispensing, building, performing builds and/or assays, moving, positioning, controlling, controlling of temperature, the control system(s), operating, and/or performing a workflow can be performed manually and/or can be automated, for example, in connection with and/or automated by using software and/or programming to perform any one or more of these functions. For purposes of this disclosure, the terms “code”, “software”, “program”, “application”, “software code”, “software module”, “module” and “software program” are used interchangeably to mean software instructions that are executable by a processor.

The present disclosure provides for a computer program comprising computer-executable instructions, which when the program is executed by a computer, cause the computer to carry out any one or more of the processes, methods, and/or algorithms according to the above. The computer-executable instructions can be programmed in any suitable programming language, including JavaScript, C, C #, C++, Java, Python, Perl, Ruby, Swift, Visual Basic, and Objective C. Also provided herein is a non-transitory computer-readable medium (or media) comprising computer-executable instructions, which when executed by a computer, cause the computer to carry out any of the processes, methods, and/or algorithms according to the above. As used in the context of this specification, a “non-transitory computer-readable medium (or media)” may include any kind of computer memory, including magnetic storage media, optical storage media, nonvolatile memory storage media, and volatile memory. Non-limiting examples of non-transitory computer-readable storage media include floppy disks, magnetic tape, conventional hard disks, CD-ROM, DVD-ROM, BLU-RAY, Flash ROM, memory cards, optical drives, solid state drives, flash drives, erasable programmable read only memory (EPROM), electrically erasable programmable read-only memory (EEPROM), non-volatile ROM, and RAM. The non-transitory computer readable media can include one or more sets of computer-executable instructions for providing an operating system as well as for implementing the processes, methods, and/or algorithms of the invention.

The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Any of the methods disclosed herein can be used with any of the compositions disclosed herein or with any other compositions. Likewise, any of the disclosed compositions can be used with any of the methods disclosed herein or with any other methods. Other embodiments of the invention will be apparent to those skilled in the art from consideration of the specification and practice of the invention.

1-3. (canceled)

4. A method comprising:
preparing one or more bioprinting construct through the deposition of one or more biomaterial or bioink in the same workflow in combination with other deposition modalities or workflow steps;
wherein cell(s) and/or spheroid(s) are printed or deposited with a degree of precision and/or patterning with a resolution of down to 1 μm;
wherein the same workflow refers to a set of computer-executable instructions for performing the dispensing and deposition without human intervention, such as continuously and automatically;
wherein the droplet dispensing comprises dispensing of pico- or nano-scale sized droplets; and
wherein in the same workflow large volumes can be dispensed such as and including large volumes relating to washing buffer, cell media, or crosslinking solution.

5. The method of claim 4, comprising the combination of bioprinting and with construct or scaffold specific targeted dosing as one of the workflow steps.

6. The method of claim 4, comprising printing, depositing and/or dispensing bioink and then printing, depositing, and/or positioning one or more cell or spheroid within the printed bioink.

7. The method of claim 6, wherein the bioink is printed separately from the cell(s) or spheroid(s).

8. (canceled)

9. The method of claim 4, comprising printing, depositing, and/or positioning one or more cell or spheroid then printing, depositing and/or dispensing one or more bioink, drug, and/or growth factor.

10. The method of claim 9, wherein the printing, depositing, and/or dispensing is performed such that a resolution in the picoliter range is achieved.

11. The method of claim 10, wherein the printing, depositing, and/or dispensing results in drop/spot sizes of about 1 micron to about 500 microns.

12. The method of claim 11, wherein the printing, depositing, and/or dispensing is performed with a precision in the micrometer range.

13. (canceled)

14. A bioprinter comprising:
one or more or multiple print heads;
one or more printbed; and
one or more liquid dispenser.

15. The bioprinter of claim 14, wherein the one or more or multiple printheads and/or the one or more printbed is moveable/positionable in x-, y-, and/or z-directions with a precision in the micrometer range.

16. The bioprinter of claim 15, wherein the precision is as low as 1 micrometer.

17. The bioprinter of claim 16, wherein the one or more printhead is capable of depositing bioink and/or spheroids.

18. (canceled)

19. The bioprinter of claim 17, further comprising a positioning arm, wherein the positioning arm is capable of performing one or more function to facilitate bioprinting, crosslinking, washing, and/or dispensing of one or more solid and/or liquid.

20. The bioprinter of claim 19, wherein the positioning arm is capable of performing one or more function to facilitate bioprinting, crosslinking, washing, and/or dispensing of one or more solid and/or liquid without user intervention.

21. The bioprinter of claim 20, wherein the one or more function is placement of a printing surface onto the printbed, or removal or placement of a lid from/on a printing surface.

22. The bioprinter of claim 14, wherein the liquid dispenser is capable of dispensing one or more liquid and/or bioink with a resolution of at least 1 picoliter.

23. The bioprinter of claim 22, wherein the bioprinter is capable of printing a construct using spheroids and/or single cells; and
wherein the bioprinter is capable of printing spheroids and/or cells separately from one another and/or separately from bioinks.

24. (canceled)

25. The bioprinter of claim 22, wherein the bioprinter is capable of dispensing growth factors, drugs, and/or therapies.

26. (canceled)

27. The bioprinter of claim 22, wherein the dispensing can be performed with precision in the micrometer range and with dispensing precision or repeatability in the range of less than 3 micrometers.

28. (canceled)

29. The bioprinter of claim 14, wherein the one or more print head, one or more liquid dispenser, and/or one or more printbed are positionable with up to 1 μm precision.

30-31. (canceled)

32. The bioprinter of claim 1434, wherein the bioprinter is capable of providing camera quality control and/or quality assurance.

33. The bioprinter of claim 14, further comprising one or more onboard display or user interface.

34. The bioprinter of claim 33, wherein the one or more onboard display or user interface is capable of displaying images and/or video from the one or more camera(s).
